(19) United States
(12) Patent Application Publication
(10) Pub. No.: US 2008/0113602 A1
(43) Pub. Date: May 15, 2008

(54) MICROBIAL RESISTANT VEHICLE AIR CONDITIONING DUCTWORK

(76) Inventors: Norman Golm, Rochester Hills, MI (US); Stephen Risch, Troy, MI (US); Richard Hoyle, Lake Orion, MI (US); Nickolaos Economou, Oxford, MI (US)

Correspondence Address: CARLSON, GASKEY & OLDS, P.C., 400 WEST MAPLE ROAD, SUITE 350, BIRMINGHAM, MI, 48009, US

(21) Appl. No.: 11/558,040

(22) Filed: Nov. 9, 2006

(51) Int. Cl. B60H 3/00; B29C 49/00; B29C 45/00; A61L 2/16

(52) U.S. Cl. 454/156; 62/244; 264/328.1; 264/500; 422/32

(57) ABSTRACT

A vehicle HVAC system includes a HVAC components such as a duct, vent, manually adjustable airflow control head, toggle or housing manufactured from a molded plastic material having an antimicrobial additive. The antimicrobial additive is preferably introduced as an additive during molding of the HVAC components that are typically injection or blow molded parts. Usage of a plastic having an antimicrobial additive continuously fights the growth of microbes such as most common bacteria, yeasts, molds and fungi that cause stains, odors and product degradation that can cause stains, odors and product degradation. The antimicrobial additive becomes an integral part of the HVAC components and therefore is maintenance free.

### BACKGROUND OF THE INVENTION

The present invention relates to a vehicle air conditioning system, and more particularly to microbial resistant vehicle air conditioning duct work.

Vehicle air conditioning systems are provided in most vehicles to maintain the passenger compartment of the vehicle at an acceptable temperature. In general, vehicle air conditioning systems include a compressor coupled to the engine that compresses a refrigerant to its liquid state. The compressed liquid refrigerant is then delivered to a heat exchanger within the air conditioning system, where it is allowed to expand and cool the exchanger. A blower forces air across the heat exchanger and into the passenger compartment of the vehicle. As the air passes through the cold heat exchanger, it is cooled and the latent heat that was contained in the air is transferred to the refrigerant within the heat exchanger. Thus, the passenger compartment receives cool air. The heated refrigerant is then passed through a radiator where it is cooled and then delivered back to the compressor where the cycle begins anew.

As warm air passes through the heat exchanger of an automotive air conditioning system to be cooled, water vapor contained in the warm air condenses on the surfaces of the heat exchanger. During normal operation of the vehicle, the water vapor that runs to the bottom of the exchanger and is drained from the air conditioning system. However, when the vehicle's engine is shut off and the air conditioner is no longer in operation, the water that has condensed on the heat exchanger begins to evaporate within the duct work of the air conditioning system and a damp dank atmosphere is created. Such an atmosphere is ideal for the growth of mold, mildew, fungus bacteria and other microbial organism within the duct work of the system. The growth of such organisms, in turn, may result in a stale and unpleasant odor within the passenger compartment itself.

Various conventional systems have attempted to address the problems of fungus and bacteria buildup within vehicle air conditioning systems. Although typically effective, conventional systems are generally relatively complex and require periodic maintenance and servicing.

Accordingly, it is desirable to provide an uncomplicated maintenance-free vehicle air conditioning system which minimizes the growth of mold, mildew, fungus bacteria and other microbial organism within the duct work of the system.

### SUMMARY OF THE INVENTION

The vehicle HVAC system according to the present invention includes HVAC components such as a duct, vent, manually adjustable airflow control head, toggle or housing manufactured from a molded plastic material having an antimicrobial additive. The antimicrobial additive is preferably introduced as an additive during molding of the typically injection or blow molded HVAC components. Usage of a plastic having an antimicrobial additive continuously fights the growth of microbes such as the most common bacteria, yeasts, molds and fungi that may cause stains, odors and product degradation.

The present invention therefore provides an uncomplicated maintenance-free vehicle air conditioning system which minimizes the growth of mold, mildew, fungus bacteria and other microbial organism within the duct work of the system.

### BRIEF DESCRIPTION OF THE DRAWINGS

The various features and advantages of this invention will become apparent to those skilled in the art from the following detailed description of the currently preferred embodiment. The drawings that accompany the detailed description can be briefly described as follows:

FIG. 1 is a functional block diagram illustrating an air conditioning system for use with the present invention;

FIG. 2 is a perspective view of a vehicle instrument panel having an air conditioning ductwork and vent arrangement for use with the present invention;

FIG. 3 is a cross-sectional view of an adjustable airflow control heads for use with the present invention;

FIG. 4 is an exploded view of an air conditioning system illustrating a multiple of components which may be utilized with the present invention.

### DETAILED DESCRIPTION OF THE PREFERRED EMBODIMENT

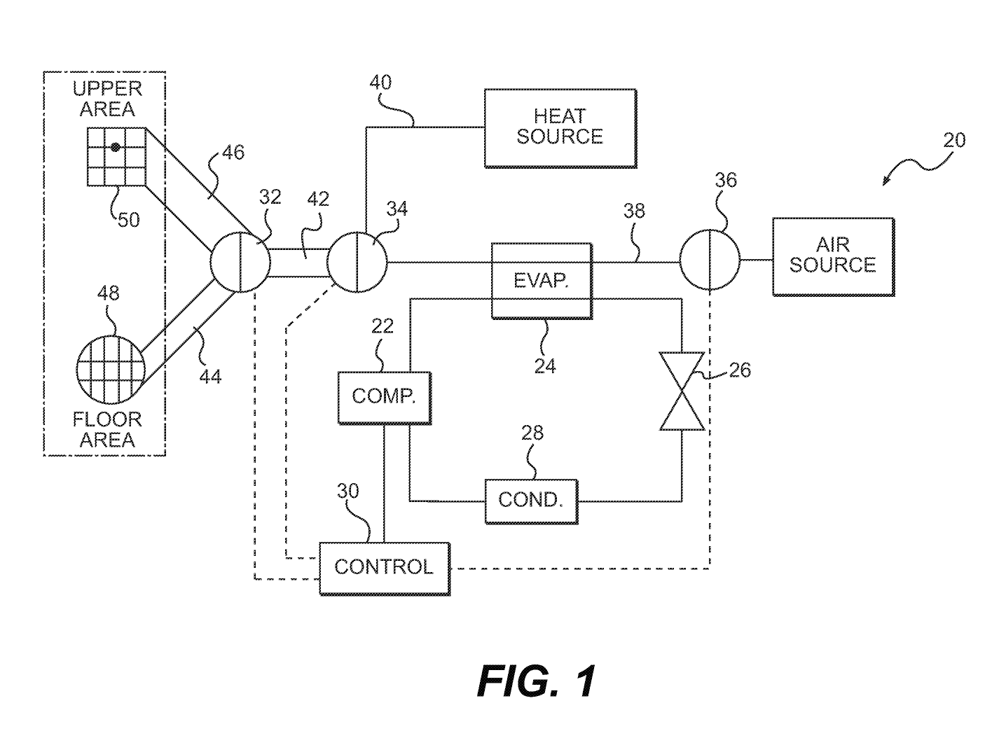

FIG. 1 schematically illustrates a portion of a vehicle HVAC system 20. As generally understood, the system 20 includes a refrigerant circuit having a compressor 22, an evaporator 24, an expansion element 26 and a condenser 28. An electronic control 30 controls the operation of at least the compressor and various fans associated with the HVAC system 20.

The HVAC system 20 includes system flow control doors 32, 34 and 36 which are preferably controlled by the control 30. The airflow flow into the vehicle passenger compartment through at least two supply channels 38, 40. As shown, supply channel 38 passes through the evaporator 24, and thus is a source of cool air. Supply channel 40 communicates from a heat source such as that generally known in vehicle HVAC systems. The two channels 38, 40 mix to reach a downstream duct 42. The door 34 controls the mixing of flow between the channels 38, 40.

Once the conditioned air reaches duct 42, it is at a generally desired temperature. Then, other doors such as flow control door 32 controls the flow between a floor area of the passenger cabin through duct 44 and the upper area of the passenger cabin through duct 46. The control 30 control preferably controls the position of the vent doors to achieve desired airflow conditions and directions within the vehicle cab. It should be understood that this is a highly schematic and simplified rendering of the overall HVAC system 20 and that there may be additional doors, flow passages, etc.

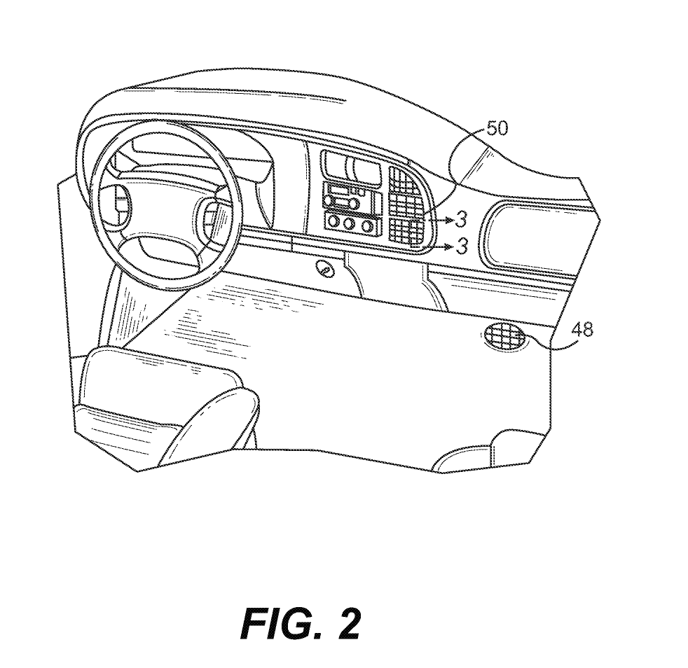

From the duct 44, the conditioned airflow is communicated through a directional vent 48 typically located within a vehicle foot well (FIG. 2). From duct 46, the conditioned airflow is communicated through a manually operated directional vent 50 typically located within the upper area of the vehicle passenger compartment (FIG. 2). It should be understood that although a particular component arrangement is disclosed in the illustrated embodiment, other arrangements will benefit from the instant invention.

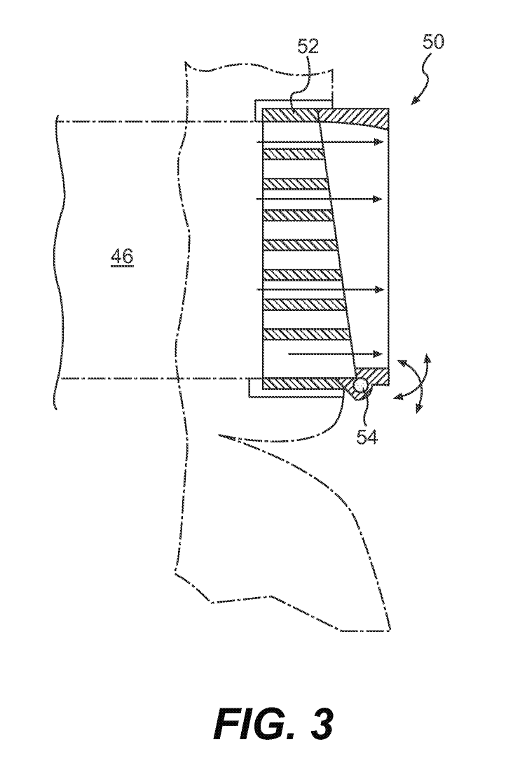

The directional vent 48 includes a grate or other airflow directing member that directs airflow in a predetermined direction. That is, the directional vent 48 is generally fixed in a single directional position. The manually operated directional vent 50 is preferably directionally adjustable to direct airflow in a desired direction by the passenger. In one type of manually operated directional vent 50 a multiple of manually adjustable airflow control heads 52 which may include pivotable slats and vanes are movable by grasping at least one toggle 54 (FIG. 3) or the like as generally understood.

Preferably, the HVAC components such as the ducts 42, 44, 46 vents 48, 50, the manually adjustable airflow control heads 52 and the toggle 54 are manufactured from a molded plastic material having an antimicrobial additive such as MICROBAN® which is produced by Microban Products Company Huntersville, N.C. USA. Although it is preferred that the components which are effectively portions of the vehicle interior such as the ducts 42, 44, 46, vents 48, 50, the manually adjustable airflow control heads 52 and the toggle 54 are manufactured from the a plastic having an antimicrobial additive, other HVAC components such as the HVAC systems component housings which surround the compressor 22, the evaporator 24, the expansion element 26 and/or the condenser 28 will also benefit from the present invention.

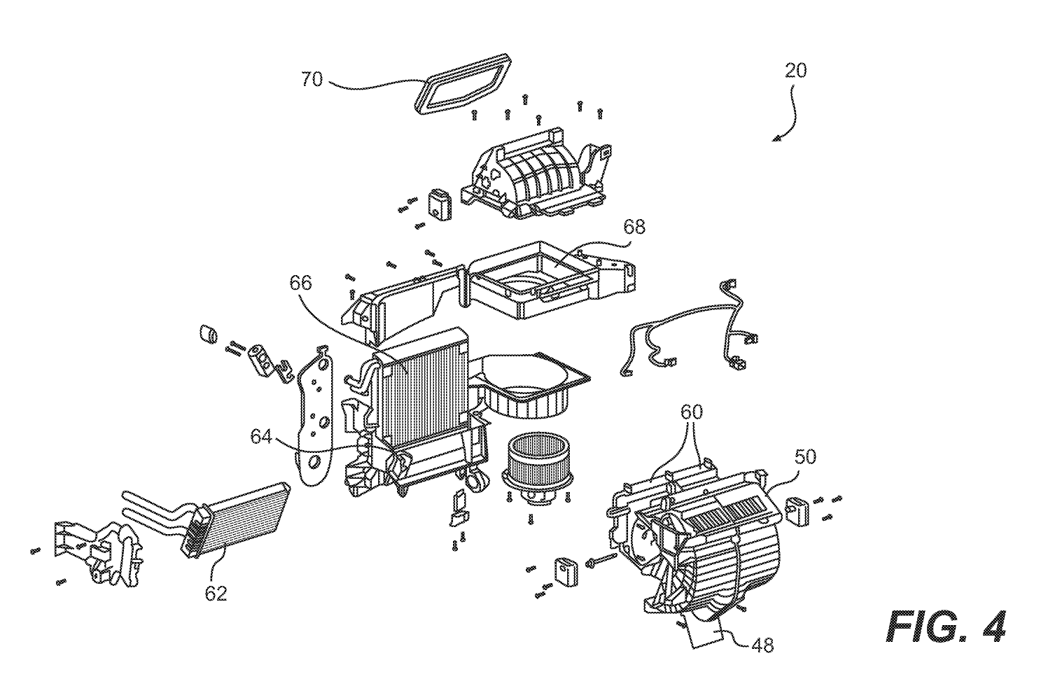

Referring to FIG. 4, other HVAC system components of the vehicle HVAC system 20 which will benefit from having an anti-microbial additive are illustrated in the exploded view. In addition to the HVAC system components described above such as events 48 and 50, HVAC cases 60, a heater core and seal 62, a sump and sump liner 64, evaporator core and seals 66, a filter 68, and a filter seal 70 may alternatively or additionally include the anti-microbial additive. It should be understood that the HVAC module illustrated in FIG. 4 is exemplary only and that these and other HVAC system components will also benefit from the present invention.

The antimicrobial additive is preferably introduced as an additive during molding of the HVAC components during typical injection or blow molding manufacturing operations. Usage of a plastic having an antimicrobial additive continuously fights the growth of microbes such as most common bacteria, yeasts, molds and fungi that cause stains, odors and product degradation. The antimicrobial additive becomes an integral molded portion of the HVAC components and therefore is maintenance free.

The foregoing description is exemplary rather than defined by the limitations within. Many modifications and variations of the present invention are possible in light of the above teachings. The preferred embodiments of this invention have been disclosed, however, one of ordinary skill in the art would recognize that certain modifications would come within the scope of this invention. It is, therefore, to be understood that within the scope of the appended claims, the invention may be practiced otherwise than as specifically described. For that reason the following claims should be studied to determine the true scope and content of this invention.

What is claimed is:

1. A HVAC system for a vehicle comprising:
a HVAC component manufactured of a molded plastic material with an antimicrobial additive.

2. The HVAC system as recited in claim 1, wherein said HVAC component includes a duct.

3. The HVAC system as recited in claim 1, wherein said HVAC component includes a vent.

4. The HVAC system as recited in claim 1, wherein said HVAC component includes airflow control head.

5. The HVAC system as recited in claim 4, wherein said HVAC component includes a vane of said airflow control head.

6. The HVAC system as recited in claim 5, wherein said HVAC component includes a toggle which controls said vane.

7. The HVAC system as recited in claim 1, wherein said HVAC component includes a HVAC component housing.

8. A method of minimizing microbial growth within a vehicle HVAC component comprising the steps of:
(1) adding an antimicrobial additive during the molding of a HVAC system component.

9. A method as recited in claim 8, wherein said step (1) further comprises:
(a) injecting molding the HVAC system component.

10. A method as recited in claim 8, wherein said step (1) further comprises:
(a) blow molding the HVAC system component.
